(19) United States
(12) Patent Application Publication
(10) Pub. No.: US 2002/0182246 A1
(43) Pub. Date: Dec. 5, 2002

(54) Device for treating and promoting healing of damaged body tissue

(76) Inventor: Michael F. Oyaski, Ebensburg, PA (US)

Correspondence Address: JAMES RAY & ASSOCIATES, 2640 PITCAIRN ROAD, MONROEVILLE, PA, 15146, US

(21) Appl. No.: 09/874,539

(22) Filed: Jun. 5, 2001

(51) Int. Cl. A61L 15/16

(52) U.S. Cl. 424/447

(57) ABSTRACT

A device for treating and promoting healing of damaged body tissue. The device comprises an encapsulating device disposed adjacent the damaged tissue for controlling the environmental conditions surrounding the tissue. A transfer device is disposed on the encapsulating device to allow at least one of a predetermined medium to communicate with the encapsulating device without removing the encapsulating device. The damaged tissue can be effectively treated and healed in controlled environmental conditions.

### FIELD OF THE INVENTION

The present invention relates, in general, to a device for treating and promoting healing of damaged body tissue, and more particularly, to a device for effectively treating and healing damaged body tissue in controlled environmental conditions.

### BACKGROUND OF THE INVENTION

Prior to the present invention, medical personnel routinely treat second and third degree burns, and skin grafts performed on burns by applying a topical antibiotic and sterile gauze bandage to the affected areas. New gauge bandages and antibiotic are applied daily. Body fluids flow from the damaged skin and dry on the bandage, making bandage removal a very painful procedure for the patient. Additionally, this procedure can interrupt and significantly slow the healing process. To prevent the affected area from drying out, the bandage must be continuously moistened. Burned tissue also feels very hot and is extremely sensitive to temperature changes. Because burns covering over 75% of the body typically result in death due to the loss of bodily fluids, the patient must be continuously rehydrated to replenish essential bodily fluids.

Medical personnel also treat severe wounds by suturing, applying antibiotics, and covering with a gauge bandage to protect the affected area during the healing process. Bodily fluids that seep from the wound adhere the gauze bandage to the wound, causing pain to the patient when the bandage is removed. In deep wound situations, standard emergency procedure is to apply a tourniquet to the affected area to restrict the loss of blood. However, a tourniquet can also damage healthy tissue by restricting the blood flow, therefore the tourniquet must be periodically loosened to prevent tissue damage and subsequent infection.

### SUMMARY OF THE INVENTION

In a first aspect, the present invention provides a device for treating and promoting healing of damaged body tissue. The device comprises an encapsulating means disposed on the tissue for controlling the environmental conditions surrounding the tissue. A transfer means is disposed on the encapsulating means to allow at least one of a predetermined medium to communicate with the encapsulating means without removing the encapsulating means. The tissue can be effectively treated and healed in controlled environmental conditions.

In a further aspect, the present invention provides a device for treating and promoting the healing of damaged body tissue. The device comprises a bladder having a predetermined size, shape, and material, and a first, and second membrane. A pressurized medium is disposed intermediate the first membrane and the second membrane of the bladder for controlling the environmental conditions surrounding the tissue, and for applying controlled pressure to the tissue. A transfer means is disposed on the bladder to allow the pressurized medium to communicate with the bladder. Controlled pressure can be applied to the tissue to promote healing.

In still a further aspect, the present invention provides a device for treating and promoting the healing of damaged body tissue. The device comprises a bodysuit of a predetermined size, shape, and material for enclosing at least a portion of a body. A transfer means is disposed on the bodysuit to allow at least one of a predetermined medium to communicate with the bodysuit without removing the bodysuit, whereby the tissue can be effectively treated and healed in controlled environmental conditions.

### OBJECTS OF THE INVENTION

It is therefore the primary object of the present invention to provide a device for treating and promoting healing of damaged body tissue.

Another object of the present invention is to provide a device for treating and promoting healing of damaged body tissue by controlling the environmental conditions surrounding the damaged body tissue.

Still a further object of the present invention is to provide a device for treating and promoting healing of damaged body tissue whereby the tissue can be effectively treated with a minimum of pain experienced by the patient.

In addition to the various objects of the invention that have been described above, various other objects and advantages of the invention will become more readily apparent to those persons skilled in the relevant art from the following more detailed description of the invention, particularly, when such description is taken in conjunction with the attached drawing figures and the appended claims.

### BRIEF DESCRIPTION OF THE DRAWINGS

FIG. 1 is a side elevational section view of the present invention;

FIG. 2 is a side elevational section view of an alternative embodiment of the present invention; and

FIG. 3 is a top view of another alternative embodiment of the present invention.

### BRIEF DESCRIPTION OF A PRESENTLY PREFERRED AND VARIOUS ALTERNATIVE EMBODIMENTS OF THE PRESENT INVENTION

Prior to proceeding to a more detailed description of the invention, it should be noted that identical components having identical functions have been designated with identical reference numerals for the sake of clarity.

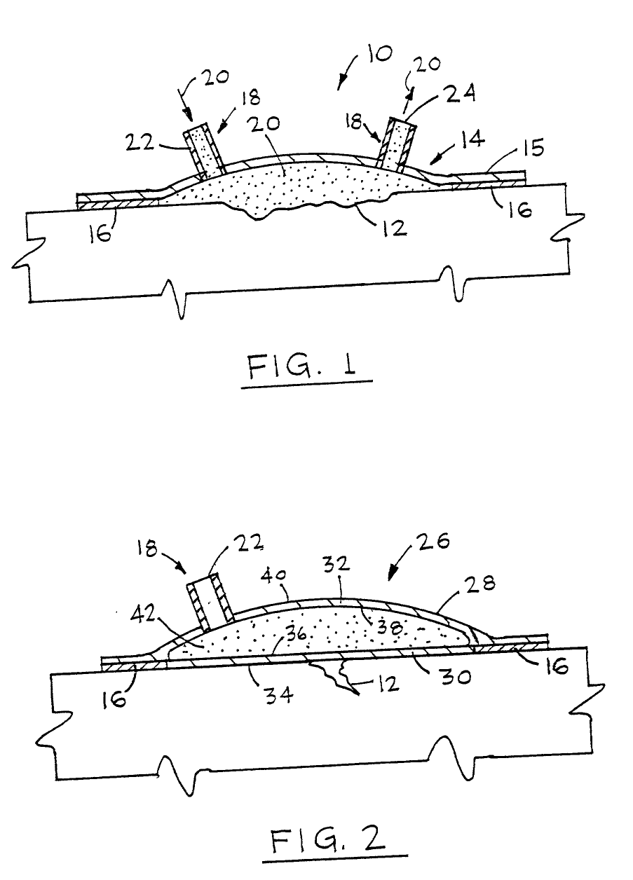

Now refer more particularly to FIG. 1 of the drawings. Illustrated therein is a device, generally designated 10, for treating and promoting healing of damaged body tissue 12. The device 10 comprises an encapsulating means 14 disposed on the body tissue 12 for controlling the environmental conditions surrounding the tissue. The encapsulating means 14 creates a sterile environment, and further allows for the temperature and humidity to be controlled for the patient's comfort, and to enhance the healing process. Preferably, the encapsulating means 14 is a bandage of a predetermined size, shape, and material. Preferably, the material is transparent, so that the damaged tissue 12 can be viewed directly through the bandage without having to remove the bandage. It is also preferable to have an adhesive 16 disposed on the perimeter of at least one side of the bandage. The adhesive 16 will secure the bandage around the damaged tissue 12, sealing the affected area from exterior environmental conditions.

The device also comprises a transfer means, generally designated 18, disposed on the encapsulating means 14 to allow at least one of a predetermined medium 20 to communicate with the encapsulating means 14 and the tissue 12 without having to remove the encapsulating means 14. Preferably, the transfer means 18 is at least one port of a predetermined size and shape. It is preferred that the transfer means 18 is at least one inlet port 22, and one outlet port 24. Preferably, the medium 20 that communicates with the encapsulating means 14 and the tissue 12 is at least one of a liquid and a gas.

Now refer more particularly to FIG. 2 of the drawings. Illustrated therein is a device, generally designated 26, for treating and healing of damaged body tissue 12. The device 26 comprises a bladder 28 having a predetermined size, shape, and material. Preferably, the bladder 28 has a first membrane 30 and a second membrane 32. The first membrane 30 further has a first surface 34 and a second surface 36, and the second membrane 32 has a third surface 38 and a fourth surface 40. The first surface 34 of the first membrane 30 is disposed on the tissue 12. Preferably, the material of the bladder 28 is transparent so that the damaged tissue 12 is visible through the bladder. It is also preferred that an adhesive 16 is disposed on the perimeter of at least one of the first surface 34 of the first membrane 30 and the fourth surface 40 of the second membrane 32, for securing the bladder 28 to the area surrounding the damaged tissue 12. Preferably, a pressurized medium 42 is disposed intermediate the second surface 36 of the first membrane 30 and the third surface 38 of the second membrane 32 for controlling the environmental conditions surrounding the damaged tissue 12, and for applying controlled pressure to the tissue 12. It is preferred that the pressurized medium 42 is at least one of a gas and a liquid. A transfer means, generally designated 18, is disposed on the bladder 28 to allow the pressurized medium 42 to communicate with the bladder 28, whereby controlled pressure can be applied to the damaged tissue 12 to seal the tissue 12 and promote healing. Preferably, the transfer means 18 is at least one inlet port 22 of a predetermined size and shape.

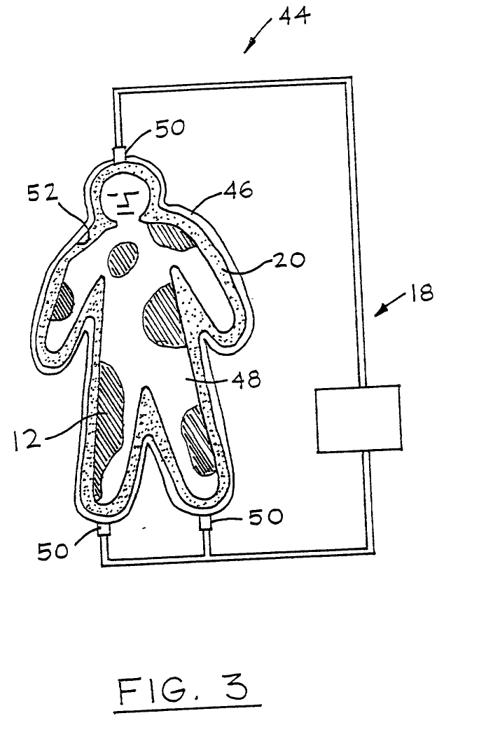

Now refer more particularly to FIG. 3 of the drawings. Illustrated therein is a device, generally designated 44, for treating and promoting healing of damaged body tissue 12. The device 44 comprises a bodysuit 46 of a predetermined size, shape, and material for enclosing at least a portion of a body 48 when larger, and multiple areas of tissue are damaged. Preferably, the material of the bodysuit 46 is transparent to allow the body to be viewed through the bodysuit 46. It is also preferable to have a texture on the inside surface 52 of the bodysuit 46 to permit fluid circulation around the body area that directly contacts the bodysuit 46 when the patient is lying down. A transfer means, generally designated 18, is disposed on the bodysuit 46 to allow at least one of a predetermined medium 20 to communicate with the bodysuit 46 without removing the bodysuit 46, whereby the tissue 12 can be effectively treated and healed in controlled environmental conditions. Preferably, the transfer means 18 is at least one port 50 of a predetermined size and shape. However, it is preferred that the transfer means 18 includes a plurality of ports 50 to control temperature, humidity, and to circulate liquids, medications, and pain killers around the body 48. It will also be obvious to the reader that a bodysuit 46 that envelops the entire body will have additional ports for feeding and the elimination of bodily waste.

### OPERATION

Damaged body tissue 12, such as a burn or wound, on a portion of the body 48 is covered with the encapsulating means 14, or bandage. The bandage is sealed around the damaged body tissue 12 by the adhesive disposed on the perimeter of the bandage. Antibiotics and other appropriate medications are injected into the inlet port 22. Inserting a suitable pharmaceutical liquid through the inlet port 22, and extracting the liquid through the outlet port 24 cleanses the affected area. Medication is then reapplied to the clean tissue 12 through the inlet port 22. The bandage remains in place throughout the healing process, maintaining a sterile, humidity and temperature controlled environment. When deeper penetration is required, medications and pain relievers can be applied under pressure to the specific area.

The bladder 28 exerts pressure on a wound, similar to a tourniquet, but in a controlled manner. The pressure in the bladder 28 can be regulated to apply the necessary force required to decrease blood loss, while decreasing the pressure at periodic intervals to prevent surrounding tissue damage and infection.

The bodysuit 46 encapsulates a larger area of damaged body tissue 12 simultaneously. Temperature and humidity can be controlled in the bodysuit 46, while medications and pain relievers can be circulated around the entire area to treat the damaged body tissue 12 and promote healing. In specific circumstances, the encapsulated area can be submerged in water to relieve the pressure on the damaged tissue. The affected area remains sterile and dry. When a full bodysuit 46 is required, the bodysuit 46 can be designed to provide breathing apparatus and elimination of bodily wastes.

Although the invention has been shown in connection with a certain specific embodiment, it will be readily apparent to those skilled in the art that various changes in form and arrangement of parts and method may be made to suit requirements without departing from the spirit and scope of the invention.

I claim:

1. A device for treating and promoting healing of damaged body tissue, said device comprising:
(a) an encapsulating means disposed adjacent such tissue for controlling the environmental conditions surrounding such tissue; and
(b) a transfer means disposed on said encapsulating means to allow at least one of a predetermined medium to communicate with said encapsulating means without removing said encapsulating means, whereby such tissue can be effectively treated and healed in controlled environmental conditions.

2. The device according to claim 1 wherein said encapsulating means is at least one of a bandage, bladder, and bodysuit of a predetermined size, shape, and material.

3. The device according to claim 2 wherein said encapsulating means is a transparent bandage of a predetermined size, shape, and material.

4. The device according to claim 3 wherein an adhesive is disposed on the perimeter of at least one side of said bandage.

5. The device according to claim 1 wherein said transfer means is at least one port of a predetermined size and shape.

6. The device according to claim 5 wherein said transfer means includes a plurality of ports.

7. The device according to claim 6 wherein said transfer means is at least one inlet port and one outlet port.

8. The device according to claim 1 wherein such medium is at least one of a liquid and gas.

9. A device for treating and promoting healing of damaged body tissue, said device comprising:
(a) a bladder having a predetermined size, shape, and material, said bladder further having a first membrane and a second membrane;
(b) a pressurized medium disposed intermediate said first membrane and said second membrane of said bladder for controlling the environmental conditions surrounding such tissue, and applying controlled pressure to such tissue; and
(c) a transfer means disposed on said bladder to allow said pressurized medium to communicate with said bladder, whereby controlled pressure can be applied to such tissue to promote healing.

10. The device according to claim 9 wherein said first membrane further has a first surface and a second surface, and said second membrane further has a third surface and a fourth surface.

11. The device according to claim 10 wherein said first surface of said first membrane of said bladder is disposed on such tissue.

12. The device according to claim 9 wherein said pressurized medium is at least one of a liquid or a gas.

13. The device according to claim 9 wherein said material of said bladder is transparent.

14. The device according to claim 9 wherein an adhesive is disposed on the perimeter of at least one side of said bladder.

15. The device according to claim 9 wherein said transfer means is at least one port of a predetermined size and shape.

16. A device for treating and promoting healing of damaged body tissue, said device comprising:
(a) a bodysuit of a predetermined size, shape, and material for enclosing at least a portion of a body; and
(b) a transfer means disposed on said bodysuit to allow at least one of a predetermined medium to communicate with said bodysuit without removing said bodysuit, whereby such tissue can be effectively treated and healed in controlled environmental conditions.

17. The device according to claim 16 wherein said material of said bodysuit is transparent.

18. The device according to claim 16 wherein the inside surface of said bodysuit is textured.

19. The device according to claim 16 wherein said transfer means is at least one port of a predetermined size and shape.

20. The device according to claim 19 wherein said transfer means includes a plurality of ports.
